(19) United States
(12) Patent Application Publication
(10) Pub. No.: US 2022/0267868 A1
(43) Pub. Date: Aug. 25, 2022

(54) ASSOCIATION BETWEEN INTEGRATION OF HIGH-RISK HPV GENOMES DETECTED BY MOLECULAR COMBING AND THE SEVERITY AND/OR CLINICAL OUTCOME OF CERVICAL LESIONS

(71) Applicant: GENOMIC VISION, Bagneux (FR)

(72) Inventors: Fanny LEMEE, Lauzerville (FR); Stephanie BOUCHILLOUX, Chatenay-Malabry (FR); Sebastien BARRADEAU, Paris (FR); Anne JACQUET, Le Plessis-Robinson (FR); Aaron BENSIMON, Bagneux (FR)

(73) Assignee: GENOMIC VISION, Bagneux (FR)

(21) Appl. No.: 17/694,264

(22) Filed: Mar. 14, 2022

Related U.S. Application Data
(63) Continuation of application No. 16/795,782, filed on Feb. 20, 2020, now abandoned.
Application No. 16/795,782 is a continuation of application No. 15/976,758, filed on May 10, 2018, now abandoned.
(60) Provisional application No. 62/504,295, filed on May 10, 2017.

(51) Int. Cl. C12Q 1/70; C12Q 1/6886

(52) U.S. Cl. CPC C12Q 1/708; C12Q 1/6886

(57) ABSTRACT

The invention involves methods for identification of biomarkers of the severity of an HPV infection.

### CROSS-REFERENCE TO RELATED APPLICATIONS

This is a Continuation of U.S. application Ser. No. 15/976,758, filed May 10, 2018, which claims priority to U.S. Provisional Application No. 62/504,295, filed May 10, 2017 both of which are incorporated by reference for all purposes. This application is also related to and Mahiet, et al., US 2016 0047006 A1, filed Mar. 4, 2015, entitled “Diagnosis of Viral Infections by Detection of Genomic and Infectious Viral DNA by Molecular Combing”; Lebofsky, et al., U.S. Pat. No. 7,985,542 B2, filed Sep. 7, 2006 entitled “Genomic Morse Code”; and Lebofsky, et al., U.S. Pat. No. 8,586,723 B2, filed Sep. 5, 2007 entitled “Genomic Morse Code”. Each of these documents is also incorporated by reference in its entirety.

### BACKGROUND OF THE INVENTION

### Field of the Invention

The invention falls within the fields of virology and molecular biology, especially as applied to medical diagnostics and therapy.

### Description of the Related Art

Cervical cancer and screening. Cervical cancer was the tenth most common cancer in teims of frequency in Czech women In 2013 there were 895 new cases for a standardized incidence rate of 11/100,000 person-years (105^{th} position in the world) with 388 deaths for a standardised mortality rate of 3.76/100,000 person-years (130^{th} position in the world).

The onset of this cancer is associated with persistent infection by one or several high-risk human papillomaviruses (HR-HPV) and has been recognised by the WHO as attributable in nearly 100% of cases to these viruses. The most common genotypes associated with cervical cancer are HPV genotypes 16, 18, 31, 33 and 45, which are responsible for more than 80% of these cancers. Because of its slow progression, cervical cancer can be prevented by screening and the treatment of the precancerous lesions that precede it. Ministry of Health of the Czech Republic initiated the National programme of the cervical cancer screening in 2008. The aim of the programme is early detection of cervical cancer. The screening is available for all women older than 15 years. The screening is currently based on the cytological examination of Pap smears once per year. In case of positive finding the cytological examination is performed again after 4 months in ASC-US or after 7 months for LSIL.

Recently, some European countries (Netherlands, Belgium) have planned to abandon cytological screening for the HPV screening test, which is considered to be more sensitive (>90%) than cytology in detecting precancerous and cancerous lesions of the uterine cervix. The slightly lower specificity of HPV testing requires the use of a triage test to avoid excessive use of colposcopy. The procedure that has been best assessed to date is a cytological screening examination. However, other triage tests that would specifically identify patients at risk of lesion progression may have their place in this screening strategy.

Integration of the HPV genome/Viral integration, a major event in tumour progression. HR-HPV infection is considered to be the major cause of cervical cancer (zur Hausen 2002). However, most infections are spontaneously cleared by the immune system, while some persist for several years and sometimes progress to cancer (Crosbie, Einstein et al. 2013). HR-HPV infection is therefore a necessary but not sufficient cause of cervical cancer. Integration of the high-risk HPV genome in the host genome is considered to be a key event in the development of cervical cancer and as one of its most important risk factors (Pett and Coleman 2007). During the initial infection phase, HPV is present as a nuclear episome; however the integration of HR-HPV DNA into the host genome is a major step in the progression of cervical neoplasia (Wentzensen, Vinokurova et al. 2004). The integration of the HR-HPV genome in the host cell genome gives these cells a strong selective advantage promoting the clonal expansion of this cell population. Three major mechanisms play an important role in this process: Firstly, it has been reported that the integration of the HR-HPV genome frequently and preferentially causes a deletion in the open reading frame (ORF) of the viral E2 gene (Choo, Pan and al. 1987). The E2 protein is a negative regulator of the viral E6/E7 promoter. One of the consequences of this integration is the loss of control of expression of viral oncoproteins E6 and E7, leading to their stable over-expression. E6 and E7 target the p53 and pRb tumour suppressors respectively, negatively regulating their anti-tumour function (Dyson, Howley et al. 1989, Scheffner, Werness et al. 1990). Secondly, the integration of HR-HPV increases the stability of the E6 and E7 viral transcripts derived from integrated viral genome copies (von Knebel Doeberitz, Bauknecht et al. 1991), thus increasing their level of expression and their oncogenicity (Jeon, Allen-Hoffmann et al. 1995, Jeon and Lambert 1995). Finally, the integrated viral genes may activate the cellular oncogenes or inactivate tumour suppressor genes close to their integration sites. This is the case, for example, for the c-Myc oncogene, the protein expression of which increases when HR-HPV is integrated in the adjacent regions (Wentzensen, Ridder et al. 2002, Ferber, Thorland et al. 2003, Peter, Rosty et al. 2006, Hu, Zhu et al. 2015). A molecular combing study showed that integration of HR-HPV at this locus was associated with a strong genetic instability of this genomic region (Herrick, Conti et al. 2005) sometimes leading to malignant progression.

Viral integration: a potential diagnostic and prognostic marker. From a morphological point of view, nothing distinguishes a lesion that will regress or persist from one that will progress to invasive cancer. In most cervical carcinomas, the HPV genome is integrated, whereas it is mainly in episomal form in low-grade lesions (Klaes, Woerner et al., 1999, Hopman, Smedts et al. 2004, Wentzensen, Vinokurova et al. 2004, Vinokurova, Wentzensen et al. 2008, Hu, Zhu et al. 2015). While it is recognised that integration of HR-HPV is detected in high-grade precancerous lesions and malignant tumours, an increasing number of studies support the idea that HPV integration may take place at a much earlier stage of cervical carcinogenesis, in lower grade lesions (Kulmala, Syrjanen et al. 2006, Cricca, Morselli-Labate et al. 2007, Huang, Chao et al. 2008, Gradissimo Oliveira, Delgado et al. 2013, Vega-Pena, Illades-Aguiar et al. 2013, Zubillaga-Guerrero, Illades-Aguiar et al. 2013, Marongiu, Godi et al. 2014, Hu, Zhu et al. 2015). Some of these studies have even demonstrated the presence of integrated HR-HPV DNA in women with normal cytology (Kulmala, Syrjanen et al. 2006, Gradissimo Oliveira, Delgado et al. 2013, Vega-Pena, Illades-Aguiar et al. 2013, Zubillaga-Guerrero, Illades-Aguiar et al. 2013, Marongiu, Godi et al. 2014). Some data also suggest that the rapid progression of early cervical lesions to high-grade lesions is closely associated with the integration of HPV 16 and in particular, a high integrated HPV16 viral load (Peitsaro, Johansson et al. 2002, Vega-Pena, Illades-Aguiar et al. 2013). Moreover, the HPV integration appears to be a risk factor for progression of precancerous CIN2-3 lesions to the cancer stage (Hopman, Smedts et al. 2004).

The detection of integration of high-risk HPV genomes in the host genome may therefore provide a useful marker to identify lesions at high risk of progression that will require treatment. It would then be possible to reduce the number of unnecessary colposcopies and the over-treatment of lesions that spontaneously regress.

Methods for detecting viruses are described by Anderson, et at, CA 2943626, filed Mar. 19, 2015, entitled “HPV16 Antibodies as Diagnostic and Prognostic Biomarkers in Pre-invasive and Invasive Disease”; Lebofsky and Bensimon, Briefings in Functional Genomics and Proteomics, vo. 1 No. 4. 385-396 (January 2003), entitled “Single DNA molecule analysis: Applications of Molecular Combing”; Raybould, et al., Journal of Virological Methods 206 (2014) 51-54, entitled HPV integration Detection in CaSki and SiHa using detection of Integrated papillomavirus sequences and restriction-site PCR”; Meoiger, et al., EP 3106524 A1, filed Oct. 14, 2013, entitled “PRDM14 and FAM19A4, Molecular Diagnostic Markers for HPV-induced Invasive Cancers and Their High-Grade Precursor Lesions”; Peitsaro, et al., J. Clinical Microbiology, Mar. 2002, p. 886-891, entitled Integrated Human Papillomavirus Type 16 is Frequently Found in Cervical Cancer Precursors as Demonstrated by a Novel Quantitative Real-Time PCR Technique”; Jansen-Durr, et al., U.S. 2016/0237143 A1, filed Jan. 6, 2016, entitled Anti-HPV E7Antibodies”; Luft, et al., Int. J. Cancer 92, 9-17 (2001), entitled “Detection of Integrated Papillomavirus Sequences by Ligation-Mediated PCR (DIPS-PCR) and Molecular Characterization in Cervical Cancer Cells”; Hu, et al., Nature Genetics, published online 12 Jan. 2015; doi:10.1038/ng.3178, entitled “Genome-wide profiling of HPV integration in cervical cancer identifies clustered genomic hot spots and a potential microhomology-mediated integration mechanism”; and Lee, et al., U.S. 2016/0376662 A1, filed Dec. 5, 2015, entitled “Improved Cervical Cancer Diagnosing Method and Diagnostic Kit for Same”.

Molecular combing procedures and Genomic Morse Code procedures are described by the cross-referenced applications above.

Due to its slow progression, cervical cancer can be prevented by screening and the treatment of the precancerous lesions that precede it. This screening is in European countries currently based on the cytological examination of cervical (Pap) smears. The national guidelines specify, for each type of anomaly, the cases in which colposcopy is indicated in order to take a biopsy sample to complete the diagnostic process. The integration of the high-risk HPV genome in the cell genome is considered to be a key event in the development of cervical cancer and as one of its most important risk factors. In most cervical carcinomas, the HPV genome is integrated, whereas it is mainly in episomal form in low-grade lesions. Other data also suggest that the rapid progression of early cervical lesions to high-grade lesions is closely associated with the integration of HPV. Detecting the integration of high-risk HPV genomes in the cellular genome may therefore provide a useful marker for the identification of high-grade lesions or lesions at high risk of progression. This would make it possible to reduce the number of unnecessary colposcopies, avoid over-treatment of lesions that spontaneously regress and better target the lesions requiring treatment. In view of the morbidity associated with cervical cancer and the unmet need for a sensitive and specific way of diagnosing, monitoring and prognosing HPV-associated diseases, the inventors investigated application of Molecular Combing techniques.

### BRIEF DESCRIPTION OF THE INVENTION

The present invention concerns the identification of biomarkers of the severity of an I IPV infection in subjects or patients. The biomarker is characterized by high number of integrations of one or more High Risk (HR) HPV DNA in genome of cervical cells of patients or subjects comprising multiple complete genomes or fragment thereof containing at least 10% of the HPV genome corresponding to the size for example of the region E6 E7 DNA. It also relates to methods and tools for the detection of integration in the genome of subjects or patients, of HPV classified as HR such as HPV 16, 18, 31, 33, 45 35, 39, 51, 52, 56, 58, 59, 66 and 68. In addition, it concerns a method of assessing the risk of having or developing a cervical cancer comprising detecting or quantifying a number of integrations of HPV DNA into, or an integration pattern of HPV DNA in, genomic host DNA obtained from a patient or subject, thereby assessing the risk of having or developing cervical cancer. Specific, but not limited, embodiments include the following:

A method for assessing a risk of having or developing a cervical cancer including detecting or quantifying a number of integrations of HPV DNA into, or an integration pattern of HPV DNA in, genomic host DNA obtained from a patient or subject, thereby assessing the risk of having or developing cervical cancer. In some embodiments of this method a greater number of instances, or a greater amount of, integrated HPV DNA is indicative of a high risk of having or developing cancer or is indicative of a more aggressive or higher grade cancer compared to a patient or subject having fewer instances or lesser amounts of integrated HPV DNA. In other embodiments of this method a different pattern of HPV DNA integrations into genomic host DNA, compared to those in a control subject or patient, is indicative of a high risk of having or developing cancer or is indicative of a more aggressive or higher grade cancer. Such risks include the risk of having, developing, or relapsing with, cervical cancer. HPV DNA from different sources may be assessed using the methods disclosed herein, including those from pathogenic strains of HPV and from those closely associated with cancer. HPV strains include strains 16, 18, 21, 31, 33, 45, 52 and 58. In other embodiments, the method may be performed with HPV DNA from a lower risk strain, such as HJPV 6 or 11 or other strains merely associated with genital warts or mild cervical abnormalities.

The methods disclosed herein may also include detecting or quantifying a number of integrations of HPV DNA by comparison to a patient prior to HPV infection or clinical signs of HPV, a subject not infected with HPV, a subject not infected with a pathogenic strain of HPV, a subject having no lesions or other symptoms of HPV infection, or a subject having substantially no antibody titer or cellular immunity to HPV or to a particular HPV strain. For example, the methods disclosed herein may include detecting or quantifying a number of integrations of HPV DNA in a patient compared to the number of integrations in earlier biological sample obtained from the same patient or subject or with regard to a HPV-negative or HPV-positive control subject.

Detecting or quantifying a number of integrations in the methods disclosed herein may be performed using molecular combing of the genomic host DNA using probes that bind to HPV DNA sequences, for example, it may be performed using molecular combing of the genomic host DNA using probes to HPV 16, 18, 31, 33, 45, 35, 39, 51, 52, 56, 58, 59, 66 and/or 68. In some embodiments, the detecting or quantifying a number of integrations is performed using molecular combing of the genomic host DNA using probes that bind to or cover HPV DNA L1 and L2, E1 and E2, and/or E6 and E7 sequences, wherein said probes may be labelled with the same or different colored fluorescent tags.

Patients assessed by the methods disclosed herein may have a cervical dysplasia or a positive PAP test. Some patients may have been previously infected with human immunodeficiency virus (HIV), may be immunosuppressed, may have been exposed to diethylstilbestrol before birth, or may have been treated for a precancerous cervical lesion or cervical cancer. In other embodiments, the methods described herein may be used to assess a risk of having anal, vaginal, vulvar, penile or oropharyngeal cancer.

In one embodiments of the method of the invention a number or pattern of HPV integrations is quantified by, or correlated, with at least one of the following: the number of HPV integration sites in host genomic DNA or the average number of such integrations; the size in kb of HPV DNA integrations into host genomic DNA; the number of HPV genomes integrated at each integration site; the presence of absence of integrated HPV DNA; the number of HPV integration sites per cellular genome; the average number of HPV integration sites in host cells; the mean number of HPV genomes integrated per integration site (or the mean size of integration sites); maximum number of HPV genomes integrated per integration site (or the maximum size of integration sites); minimum number of HPV genomes integrated per integration site (or minimum size of integration sites), or number of HPV genomes integrated per cellular genome.

In other embodiments of the methods disclosed herein, the number or pattern of HPV integrations is correlated with at least one parameter of lesion status including: normal histology (including all abnormalities without intraepithelial lesions or signs of viral infection such as metaplasia, cervicitis, decidual lesions or adenosis); low grade (LG) lesion, corresponding to former CIN1; high grade (HG) lesion, corresponding to former CIN2, 3 and CIS (carcinoma in situ) or AIS (adenocarcinoma in situ); normal cervix; Grade 1 atypical transformation (AT); Grade 2 atypical transformation (AT); TAG2 a if there are no major signs; TAG2 b if there are major signs; TAG2 c when the appearance is suggestive of invasive cancer; and/or atypical transformation (minor or major).

In other embodiments of the methods disclosed herein, the number or pattern of HPV integrations is correlated with at least one parameter of cytological classification selected from negative for intraepithelial lesion or malignancy; presence or absence of abnormal squamous cells; presence or absence of typical squamous cells (ASC) of undetermined significance (ASC-US); an inability to exclude high-grade squamous intraepithelial lesion (ASC-H); presence or absence of low-grade Squamous Intraepithelial Lesion (LSIL); presence or absence of high-grade Squamous Intraepithelial Lesion (HSIL); presence or absence of squamous cell carcinoma; presence or absence of abnormal glandular cells; atypical glandular cells (AGC): endocervical not otherwise specified or commented); endometrial or not otherwise specified; atypical glandular cells, favor neoplastic: endocervical or not otherwise specified, presence or absence of endocervical adenocarcinoma in situ (AIS) and/or adenocarcinoma that is not endocervical, endometrial, extrauterine or not otherwise specified.

In some embodiments of the method of the invention, the number or pattern of HPV integrations is correlated with clinical outcome of cervical lesions in patients including progression, stability or regression. In others, the number or pattern of HPV integrations is correlated with viral clearance, HPV vaccination status, amelioration of symptoms of HPV infection or cure or evaluation of the performance of pharmaceutical treatment or personalized treatment.

Another aspect of the invention is a visualized DNA pattern obtained after hybridization of HR-HPV DNA labelled probes with a genomic DNA of a subject or a patient suspected to contain one or multiple integrated HR-HPV genomes or specific fragments thereof said DNA pattern comprising HR-HPV DNA and genomic DNA from normal or cancerous cells. This DNA pattern may constitute HR-HPV DNA which is chosen among the genotypes 16, 18, 31, 33, 45, 35, 39, 51, 52, 56, 58, 59, 66 or 68. It may also constitute HR-HPV DNA that contains all or part of the E6 E7 DNA regions. A DNA pattern may function as a biomarker of HPV infection and integration.

### BRIEF DESCRIPTION OF THE FIGURES

This application file contains at least one drawing executed in color. Copies of this patent application publication with color drawing(s) will be provided by the Office upon request and payment of the necessary fee.

FIG. 1. Integration yes/no: High Grade (HG) compared to the normal group (p=0.0006); Low Grade (LG)+HG compared to the normal group (p=0.0012).

FIG. 2 shows the number of integrations/genome in the High Grade (HG) compared to the normal group (p=0.0001).

FIG. 3. Description of the analysis of samples by Molecular Combing.

FIG. 4. Physical principle of molecular combing. Examples of YOYO-1-stained DNA molecules, combed at different densities, visualised under a fluorescence microscope at 40× magnification.

FIG. 5A-5B together form a design diagram.

FIG. 6. HPV 18 genome coverage by the 3 probes: one covers the region containing genes L1 and L2, the second viral genes E1 and E2, and the third viral oncogenes E6 and E7. The probes of the other thirteen genotypes (HPV 16, 31, 33, 45, 35, 39, 51, 52, 56, 58, 59, 66 et 68) are drawn on the same model. The probes corresponding to regions L1L2 and E1E2 are displayed in blue and the one corresponding to region E6E7 in cyan.

FIG. 7A. Example of signal corresponding to a single integration of an HPV 16 genome. FIGS. 7A-7D exemplify signals corresponding to integrated HPV genomes and reference signals.

FIG. 7B: 4 examples of signals corresponding to 4 integration sites, each containing multiple integrations of juxtaposed HPV 16 genomes. The cyan signals, corresponding to probe E6E7, make it possible to visually count the number of integrated HPV genomes per integration site;

FIG. 7C: 3 examples of signals corresponding to 3 integration sites, each containing multiple integrations of dispersed HPV 18 genomes, spaced by the host DNA.

FIG. 7D: Examples of reference signals generated by the probes covering a defined DNA locus of known size.

FIG. 8. Diagram of types of signals detected on the slides.

### DETAILED DESCRIPTION OF THE INVENTION

HPV refers to human papilloma virus. Human papillomavirus (HPV) is a group of viruses that are extremely common worldwide. There are more than 100 types of HPV, of which at least 13 are cancer-causing (also known as high risk type). Two HPV types (16 and 18) cause 70% of cervical cancers and precancerous cervical lesions. Vaccines against HPV 16 and 18 have been approved for use in many countries.

HPV DNA includes the polynucleotides of the HPV genome, individual genes of the HPV genome, as well as fragments of the HPV genome or genes, such as polynucleotide fragments that are recognized by probes to HPV polynucleotide sequences.

Control Subject or Patient. Those skilled in the art are aware of the value of controlled comparisons. A control subject or patient includes a subject who has not been exposed to HPV, has no symptoms or indicia of HPV infection, has no or a lower antibody titer to HPV, who exhibits a lower cellular response to HPV, or who exhibits fewer or no integrations of HPV DNA into genomic DNA, than that of a patient being evaluated. Positive control patients or subjects include those known to have integrated HPV DNA in their genomic DNA, who exhibit symptoms of HPV infection, and/or who have HPV related cancers or conditions, such as those cancers or conditions described herein.

Molecular Combing has its conventional meaning as described in the applications, publications and other documents incorporated by reference herein. Some current techniques for the detection of HPV integration are described below.

Routine techniques for the diagnosis of HR-HPV infection (detection of HPV DNA or E6E7 mRNA) and genotyping make it possible to demonstrate a persistent infection or to assess the presence of multi-infections and estimate the specific prevalence of types. However, they give an indirect picture of the oncogenic process and none can describe the genomic integration of HR-HPV DNA in the cellular genome. Techniques for detecting the integration of HR-HPV are currently only used for research purposes and all have limitations in this application.

Integration of HR-HPV can be directly detected in cells by in situ hybridisation (ISH) or by fluorescence in situ hybridisation (FISH), frequently used in cytogenetics. Integrated and episomal forms can be distinguished by the diffuse (episomal) or punctuate (integrated) signal pattern. The interpretation of these patterns is partly subjective and therefore generates a variability of results between operators. Moreover, the resolution of these techniques is limited to 1-5 Mb (megabase) due to the steric hindrance of condensed DNA molecules and does not allow fine analysis of this integration (Lebofsky and Bensimon 2003). Southern blotting is also used to analyse the integration of HR-HPV. Although this method is relatively reliable, it is cumbersome and the use of radiolabelled probes has implications for the safety of operators. Integration can also be analysed by the detection of viral-cellular fusion transcripts, as is the case with the amplification of papillomavirus oncogene transcript (APOT) technique (Klaes, Woerner et al. 1999). However, this method requires extraction and amplification of mRNA which is much less stable than DNA. Moreover, it introduces bias, since it detects only transcriptionally active HPV DNA and it has been shown that in the CaSki cell line, which has a large number of genomes integrated in tandem, few seem to be actively transcribed (Van Tine, Knops et al. 2001, Ziegert, Wentzensen et al. 2003). Another technique is also based on the detection of the fusion of human and viral sequences, but at the level of DNA: DIPS (Detection of Integrated papillomavirus Sequences) (Luft, Klaes et al. 2001). However, this method is limited by its lack of sensitivity in the detection of integration when there are high concentrations of episomal forms and tandem integrations (Raybould, Fiander et al. 2014). The real-time PCR technique has these same limitations. This method determines the ratio between the number of copies of the viral E2 gene and the viral oncogene E6 (Peitsaro, Johansson et al. 2002). It is based on the fact that when the HPV genome is integrated, E2 is deleted in the vast majority of cases and E6 conserved. However, it was recently shown that cleavage during integration could occur at any site on the HPV genome, including in E6 and predominantly in El (Hu, Zhu et al. 2015). All integrations resulting from a deletion outside E2 and all tandemly integrated forms which conserve intact HPV genomes are not therefore detected by this technique. Finally, the HIVID technique (High-throughput Viral Integration Detection) based on high-throughput (new-generation) sequencing (NGS) combined with computer analysis of reads, makes it possible to precisely locate HPV integration sites (Hu, 2015.1). However, this technique is cumbersome and complicated and cannot be used to determine the number of copies integrated at a locus.

All these techniques have been used to increase our understanding of the role of HPV integration in precancerous lesions and cervical cancers. The diversity of these techniques and their limitations explain the variability and sometimes divergence between the data obtained in this field as most of these techniques do not provide an unbiased, sensitive and high-resolution analysis of the integration loci.

### EXAMPLE 1

### Association Between Integration of High-Risk HPV Genomes Detected by Molecular Combing and the Severity and/or Clinical Outcome of Cervical Lesions

A clinical study was set-up to study the association between the integration of high-risk HPV genomes detected by molecular combing and the severity of cervical lesions in patients with an indication for colposcopy after an abnormal Pap smear. An interim analysis was done on 126 patients HPV-HR. The parameters of integration determined in the interim analysis and the parameters of the lesion describing the lesion status are described below.

Integration Pattern. The integration pattern may be defined by the following variables with values that are directly determined from the above data: (i) presence or absence of integration or (ii) a number of HPV integration sites per cellular genome.

Lesion status. The results of biopsies under colposcopy and conisation specimens are given according to the new WHO classification published in 2014 include (i) normal histology (including all abnormalities without intraepithelial lesions or signs of viral infection such as metaplasia, cervicitis, decidual lesions or adenosis.), (ii) Low grade (LG) lesion, corresponding to former CIN1, (iii) High grade (HG) lesion, corresponding to former CIN2, 3 and CIS (carcinoma in situ), and (iv) AIS (adenocarcinoma in situ).

If there is a discrepancy between the histological data from biopsies performed under cervical colposcopy and the histology data from conisation specimens, the worst-case histological result will be taken into account. The group “smear-colposcopy only: describes those where neither biopsy, nor conisation have been performed.

Results of the interim analysis. The first data show that the % of patients with HPV integration and the number of integrations per genome is inferior in the normal group (abnormal cytology but normal biopsy) compared to the other groups. For example, according to the molecular combing technology, the percentage of patients with the DNA HR-HPV integration in the cancer group is 100% although in the normal group the percentage of subjects with the DNA HR-HPV is about 70%.

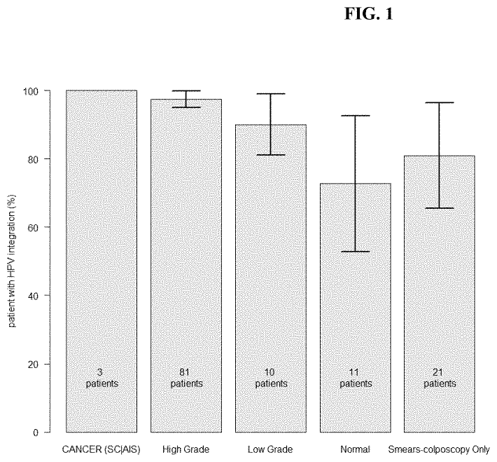

As shown by FIG. 1, the proportion test. a chi-square test with a Yates's correction for continuity is used; see Yates, F (1934). “Contingency table involving small numbers and the χ2 test”. Supplement to the Journal of the Royal Statistical Society 1 (2): 217-235. JSTOR 2983604, incorporated by reference.

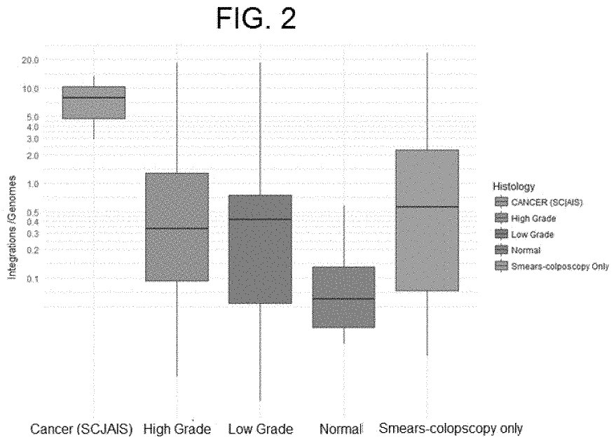

As shown by FIG. 2, a Mann-Whitney-Wilcoxon test was used to compare the variable in two independent samples that were selected from populations having the same distribution; see David F. Bauer, J. Am. Statistical Assoc. 67 (339): 687-690 (1972, incorporated by reference. Confidence sets are constructed using rank statistics.

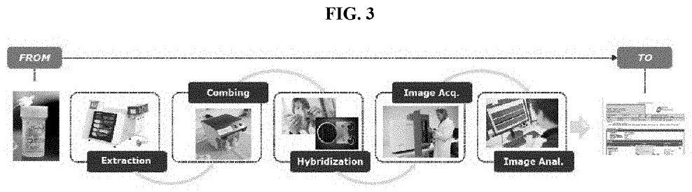

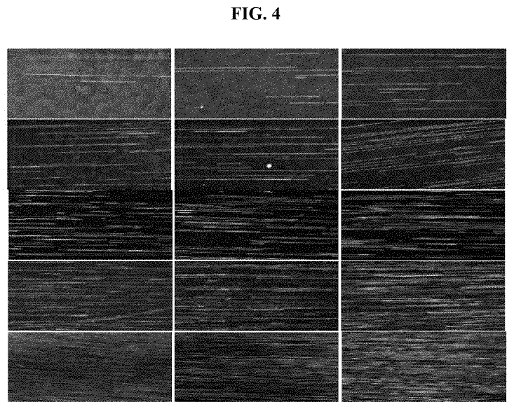

Contribution of the Molecular Combing (MC) Technique to the study of HR-HPV integration. The objective of the scientific program is to study the rare and complex genetic event that is viral integration, using a sensitive, unbiased and high-resolution technique. None of the current methods mentioned above fully meets these criteria. FIG. 3 describes analysis and workflow of samples by Molecular Combing. The first step consists in extracting very high molecular weight DNA from a biological specimen (blood, smear, etc.). Once extracted, the DNA molecules are “combed”, i.e., attached by their ends to a silane-coated glass slide and uniformly stretched by a receding air-water interface (Bensimon 1994). Images of combed DNA fibers are shown by FIG. 4.

Once they are irreversibly fixed in this configuration to the glass substrate, DNA molecules are hybridised with a set of fluorescent probes specific for the DNA sequences of interest in order to obtain the specific fluorescent signature of this DNA region. During this study, the probes used will be specific for HPV 16, 18, 31, 33, 45, 35, 39, 51, 52, 56, 58, 59, 66 and 68 or other HPV genomes, and a reference region in order to normalize the result with respect to the number of cellular genomes combed on the coverslips. Only HPV forms integrated in the cellular genome will be combed and analysed. Circular episomal forms are not analysed as they have no free DNA terminal to be combed.

After hybridisation, the slide is placed in a scanner in order to acquire images by epifluorescence microscopy corresponding to all the fields of view of the slide. Using specific software developed by Genomic Vision and commercialized for other applications under the trademark Fibervision 2016, it is possible to identify, among the thousands of fields of view, the regions of interest on the coverslip containing a fluorescent signal and to measure the size of these different signals. This last step is made possible by the presence of a constant stretching factor (2 Kb/μm) which guarantees the determination of the physical distances within the region studied, by direct measurement of the probes and their spacing.

This approach can be used to study viral integration (i) with a high resolution, of approximately one kilobase (kb), including tandem integrations, (ii) directly without initial amplification of the genetic material and therefore with no selection bias associated with a choice of primer (iii) independently of viral transcription, and (iv) in a quantifiable and objective manner None of the current and conventional analytic techniques mentioned above have all of these characteristics allowing complete, reliable and fine analysis of the integration of HR-HPV genomes in the cell genome.

### EXAMPLE 2

### Clinical Study Protocol

This protocol involves the association between Integration of High-Risk HPV Genomes Detected by Molecular Combing and the Severity and/or Clinical Outcome of Cervical Lesions. The study is indicated for patients with an abnormal cervical uterine smear undergoing diagnostic colposcopy. It is an exploratory study and involves DNA combing. The study is performed in compliance with ICH GCP.

Background and Rationale: The occurrence of cervical cancer is associated with persistent infection by one or more high-oncogenic risk human papillomaviruses (HPV). The most common genotypes associated with cervical cancer are HPV genotypes 16, 18, 31, 33 and 45, which are responsible for more than 80% of these cancers.

Because of its slow progression, cervical cancer can be prevented by screening and the treatment of the precancerous lesions that precede it. This screening is currently based on the cytological examination of cervical (Pap) smears. In the case of abnormal findings, the colposcopy is indicated in order to take a biopsy sample to complete the diagnostic process.

The integration of the high-risk HPV genome in the cell genome is considered to be a key event in the development of cervical cancer and as one of its most important risk factors. In most cervical carcinomas, the HPV genome is integrated, whereas it is mainly in episomal form in low-grade lesions. Other data also suggest that the rapid progression of early cervical lesions to high-grade lesions is closely associated with the integration of HPV. Detecting the integration of high-risk HPV genomes in the cellular genome may therefore provide a useful marker for the identification of high-grade lesions or lesions at high risk of progression. This would make it possible to reduce the number of unnecessary colposcopies, avoid over-treatment of lesions that spontaneously regress and better target the lesions requiring treatment.

One objective of the inventors was to investigate the association between the integration of high-risk HPV genomes detected by molecular combing and the severity of cervical lesions in patients with an indication for colposcopy. Other objectives include investigating the association between integration of high-risk HPV genomes detected by molecular combing and viral clearance as well as the association between the integration of high-risk HPV genomes detected by molecular combing and the clinical outcome of cervical lesions. It also involves investigating the integration rate detected by molecular combing according to the type of high-risk HPV.

Methodology. The study is designed as multicenter cohort study with prospective inclusion. The patient population studied is all women all women aged 25 to 65, consulting a Department of Obstetrics and Gynaecology participating in the research for colposcopy indicated after an abnormal Pap test (ASC-US, ASC-H, atypical glandular cells, LSIL, HSIL).

Participation in the research is proposed to all women eligible to take part in this study during a gynecology visit for colposcopy indicated after an abnormal Pap test. Patients agreeing to participate in the research (signed informed consent form) will be included in the study.

Cross-sectional part of the study (baseline visit). A cervical smear is collected and HPV genotyping is performed on this sample. Patients with a negative result for high-risk HPV genotypes will leave the study. For patients with a positive HPV result for any high-risk HPV genotype(s), the study of the integration of the HPV genome by molecular combing is performed using this sample. Photographs of the cervix are taken with a video colposcope. Appropriate management (regular monitoring or treatment of lesions) will be proposed to the patient by the gynecologist using the colposcopy results and, if appropriate, the histological analysis of biopsies performed by colposcopy.

Longitudinal part of the study. Patients for whom follow-up is proposed will participate in the longitudinal part of the study. Follow-up visits at 6, 18 and 30 months are made and involve collection of a cervical smear sample for cytological analysis. Cervical smear samples are also collected at 12, 24 and 36 months for HPV genotyping and for patients with a positive HPV result for any high-risk HPV genotype(s), the study of the integration of the HPV genome by molecular combing will also be performed using the cervical smear sample. Colposcopy, cervical images are taken with a video colposcope. Biopsies are performed when they are considered necessary by the gynecologist. For patients whose care consists of treating the lesion, participation in the study is discontinued. For patients who have become negative for high-risk HPV and the lesion regressed (confirmed in 2 consecutive visits), the participation in the study is discontinued. Pregnancy is also a reason for discontinuation during the longitudinal part of the study.

Statistical Analysis. Test for factors associated with the severity of cervical lesions, including various integration parameters of high-risk HPV genomes by univariate (Student's test, Wilcoxon, Chi2 or Fisher exact tests) and multivariate analysis (logistic regression). Test for factors associated with viral clearance, including various integration parameters of high-risk HPV genomes by univariate (Student's t test, Wilcoxon, Chi2 or Fisher exact tests) and multivariate analysis (logistic regression). Test for factors associated with the progression of cervical lesions (regression or progression), and in particular different parameters of integration of high-risk HPV genomes by univariate (Student's test, Wilcoxon, Chi2 or Fisher exact tests) and multivariate analysis (logistic regression). One interim analysis is done with accumulating cross-sectional data upon having about 100 subjects enrolled in the smaller group. The main goal of the interim analysis is a sample size re-estimation. There are no formal stopping rules. A possible inflation of type I error rate will not be adjusted for because of the exploratory design of the study.

Exclusion/Inclusion Criteria. Inclusion criteria include women 25 to 65 years of age visiting the site to undergo colposcopy in the context of an abnormal cervical uterine smear (ASC-US, ASC-H, glandular anomalies, LSIL, HSIL) performed at least one month and at most 6 months before agreeing to participate in the study as well as written consent. Exclusion criteria include those vaccinated against HPV treated for a cervical disorder with normal cytology follow-up for less than 2 years, with a known positive HIV test, with a chronic disease generating immunosuppression, with immunosuppression treatment in progress, with general corticoid treatment for 2 weeks or longer in the last 6 months, pregnant, and those with participation in a clinical trial with investigational drugs within the last 3 months before the enrolment or during the present trial period.

Evaluation. Participants are evaluated for Integration of HPV. This includes integration (presence/absence), number of HPV integration sites per cellular genome, mean number of HPV genomes integrated per integration site (or the mean size of integration sites), maximum number of HPV genomes integrated per integration site (or the maximum size of integration sites), minimum number of HPV genomes integrated per integration site (or minimum size of integration sites), number of HPV genomes integrated per cellular genome, lesion status, viral clearance, clinical outcome, and cure.

The primary objective of this investigation is to study the association between the integration of high-risk HPV genomes detected by molecular combing and the severity of cervical lesions in patients with an indication for colposcopy after an abnormal Pap smear. Other objectives include assessment of patients who underwent colposcopy for an abnormal Pap test and for whom simple monitoring is indicated: the association between the integration of HR-HPV genomes detected by molecular combing and viral clearance; the association between the integration of HR-HPV genomes detected by molecular combing and the clinical outcome of the cervical lesions; and assessment of the integration rate detected by molecular combing for each type of HR-HPV.

Integration of HPV genomes studied by molecular combing. For each patient, the parameters assessed during the analysis by molecular combing are: the estimated number of combed cell genomes on the coverslip. This number of combed cellular genomes varies from one coverslip to another, depending on the amount of DNA extracted and the combing density. It is therefore used for the standardisation of results. The number of HPV integration sites on the coverslip. The size in kilobase (kb) of HPV genome integrations. The number of HPV genomes integrated at each integration site. The integration pattern is defined by the following variables with values that are directly determined from the above data: integration (presence/absence), number of HPV integration sites per cellular genome, mean number of HPV genomes integrated per integration site (or the mean size of integration sites), maximum number of HPV genomes integrated per integration site (or the maximum size of integration sites), minimum number of HPV genomes integrated per integration site (or minimum size of integration sites), number of HPV genomes integrated per cellular genome. These values will be calculated: on all detected HPV signals, then only on HPV signals above 10 kb. This signal selection overcomes the problem of the potential contamination by viral episomal forms that may be linearized during handling, thereby allowing these forms to be combed by their free ends.

Lesion status. The results of biopsies under colposcopy and conisation specimens are given, whenever possible, according to the new WHO classification published in 2014: normal histology (including all abnormalities without intraepithelial lesions or signs of viral infection such as metaplasia, cervicitis, decidual lesions or adenosis.), low grade (LG) lesion, corresponding to former CIN1, high grade (HG) lesion, corresponding to fanner CIN2, 3 and CIS (carcinoma in situ), AIS (adenocarcinoma in situ). Nevertheless, it is possible to continue using the WHO 2003 classification (normal cervix, condyloma, CIN1, CIN2, CIN3, AIS). If there is a discrepancy between the histological data from biopsies perfoimed under cervical colposcopy and the histology data from conisation specimens, the worst-case histological result will be taken into account.

French terminology: For this study, the French and international terminologies will be used for colposcopic classification: normal cervix: includes ectropions, metaplasia and Nabothian cysts. Grade 1 atypical transformation (AT): corresponds to a centripetal area of re-epithelialisation (like normal metaplasia) but with dystrophic epithelium not producing glycogen. This area will appear as: non-congestive on examination without preparation, slightly acetowhite with sharp borders not containing any crypt openings and iodine negative with sharp borders after application of Lugol's iodine solution. Grade 2 atypical transformation (AT) defined on examination without preparation by the presence of an area of congestion, then an intense acetowhite area, with blurred margins, with the presence of crypt openings and iodine negative appearance with blurred margins after application of Lugol's iodine solution. This lesion complex presents a centrifugal progression both towards the ectocervix and endocervix showing its dysplastic nature. This grade 2 AT has several stages of increasing severity according to the number of signs of severity on the images (vascular erosions, ulcerations, vegetation, necrosis etc.). TAG2 a if there are no major signs, TAG2 b if there are major signs, and TAG2 c when the appearance is suggestive of invasive cancer. For each colposcopic appearance: normal, TAG1 or TAG2, the level of the squamocolumnar junction (SCJ) will be specified: visible or not visible. When the SCJ is not visible, colposcopy is considered non-contributory.

International terminology: This is mainly centred on the examination without preparation and after application of acetic acid as Lugol's iodine solution is not an integral and systematic part of colposcopic examination (especially for English-speaking practitioners). Normal cervix: including all aspects of normal cervix in the French terminology. Atypical Transformation: corresponds to the appearance of an acetowhite area in the transformation zone (i.e. between the original SCJ and the new SCJ: repair zone). This atypical transformation (AT) may be: minor few acetowhite areas, without any sign of seriousness corresponding to mainly CINI lesions but also including grade 1 atypical transformation considered here as a minor appearance of acetowhitening major: more marked acetowhitening, with areas with additional signs that may correspond to mainly CIN2+ lesions. For each colposcopy appearance: the level of the squamocolumnar junction (SCJ) will be specified: visible or not visible. When the SCJ area is not visible, colposcopy is considered non-contributory.

Cytological classification. Cytological analysis of cervical smears is reported using the Bethesda system terminology (2001). This provides information about: the type of specimen: conventional cervical smear, monolayer, the quality of the specimen: satisfactory or not for analysis, general classification: presence/absence of abnormal epithelial cells.

Interpretation/Result: Negative for intraepithelial lesion or malignancy, Abnormal squamous cells, Atypical squamous cells (ASC), Of undetermined significance (ASC-US), Cannot exclude high-grade squamous intraepithelial lesion (ASC-H), Low-Grade Squamous Intraepithelial Lesion (LSIL), High-Grade Squamous Intraepithelial Lesion (HSIL), Squamous cell carcinoma, Abnormal glandular cells, Atypical glandular cells (AGC): endocervical (not otherwise specified (NOS) or commented), endometrial or not otherwise specified, Atypical glandular cells, favor neoplastic: endocervical or not otherwise specified, Endocervical adenocarcinoma in situ (AIS). Adenocarcinoma: endocervical, endometrial, extrauterine or not otherwise specified.

Clinical outcome of cervical lesions in patients with simple follow-up. The clinical outcome of cervical lesions will be evaluated solely for patients participating in the longitudinal part of the study. The outcome of histological lesions discovered during an abnormal smear for which surveillance was decided will be assessed according to colposcopic, cytological and histological criteria. The three possibilities are progression, stability or regression. In case of a discrepancy, the worst-case endpoint will be taken into account.

Viral clearance. Viral clearance is evaluated solely for patients participating in the longitudinal part of the study. Viral clearance will be evaluated in 2 ways: HR-HPV clearance: All high-risk HPV detected at each visit will be considered globally. Thus, HR-HPV viral clearance will be defined by the absence of any HR-HPV during a follow-up visit and by specific type clearance: Each HR-HPV will be considered independently. Clearance of a specific viral type will be defined by a negative test during a monitoring visit for a HPV subtype present at baseline. The HR-HPV subtypes not present at baseline which could be detected during the follow-up will not be taken into account.

Cure. Cure is defined by complete regression of cervical lesions at 2 consecutive follow-up visits and clearance of HR-HPV.

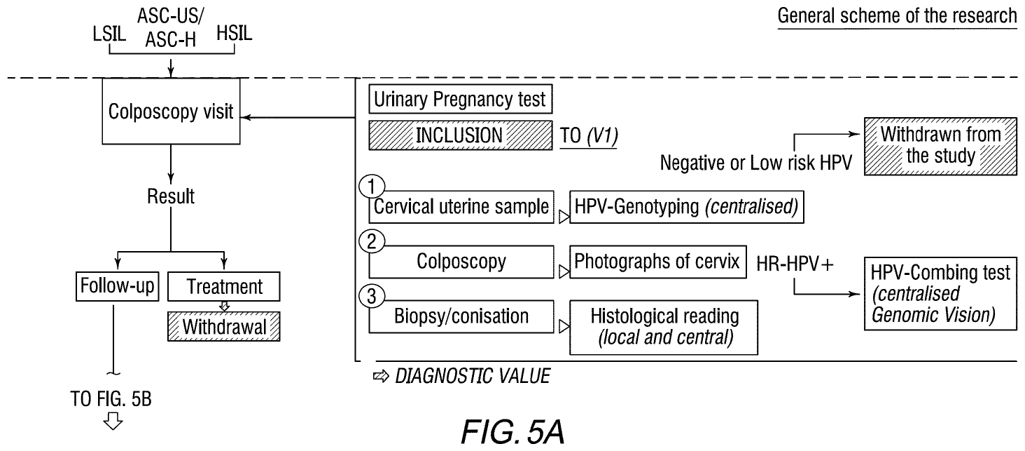

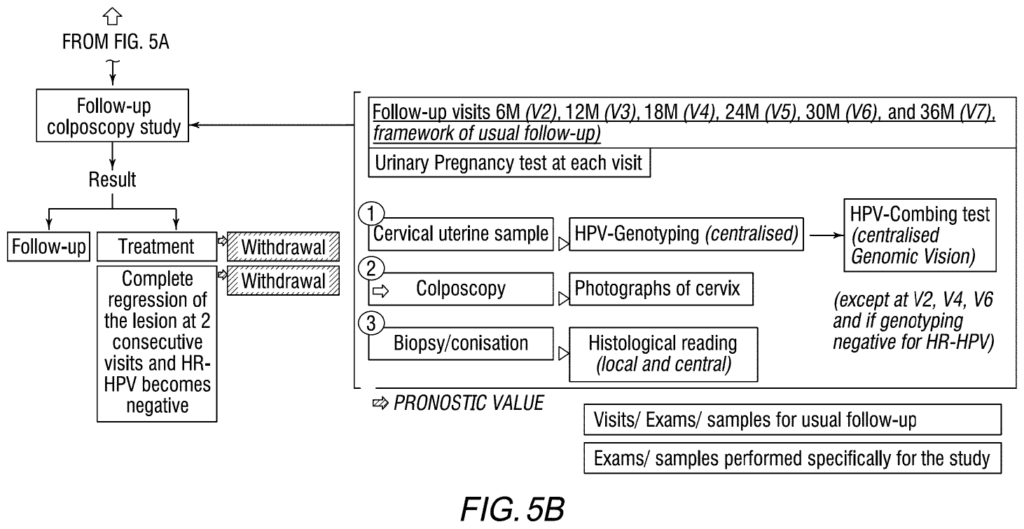

Investigational Plan. FIGS. 5A and 5B_provide a study design diagram. The study is open-label, single arm, multi-centre exploratory study with two parts: cross-sectional part (one study visit) and a longitudinal part (follow-up) — up to 6 visits during 36 months

The estimated number of patients is 993 enrolled/655 evaluable patients. The sample size will be re-calculated based on an estimation of the accumulated data parameters upon having about 100 subjects enrolled in the smaller severity group (whichever it is).

The estimated enrolment period is 6 months until the interim analysis followed by 6 months of further enrolment (the duration of the enrolment will depend on the sample size re-calculation). The duration of a patient's study participation will vary according to patient care: if the decision is follow-up with regular visits: the patient's participation will last at least 6 months and no more than 3 years, according to the decision to treat or recovery, or if the decision is to treat: the patient's participation will be one-time (no follow-up) and will consist only of the initial colposcopy visit.

To meet the main objective, two groups of patients I evaluated: One group of patients with abnormal cervical uterine smears, positive HR-HPV genotyping and a normal histology result or a low-grade histological lesion. One group of patients with abnormal cervical uterine smears, positive HR-HPV genotyping and a high-grade histological lesion.

Interim Analysis. One interim analysis will be done with accumulating cross-sectional data upon having about 100 subjects enrolled in the smaller group (whichever it is). The main goal of the interim analysis is a sample size re-estimation, however all main variables will be analysed too. There is no independent data monitoring committee to assess the interim outcome. The interim statistical report is available to the Sponsor directly; no data assessment meeting is planned. There are no formal stopping rules. Nevertheless, based on the interim results the sponsor will decide whether to continue or discontinue, seek extra data, and/or make modification of the study design. Unless this happens, however, the principal investigators and central administrative staff will remain ignorant of the interim results of the accumulated data. For the statistical considerations of the interim analysis see the statistical section of this study protocol.

Cervical uterine sample management at M0, M12, M24 and M36. The cervical uterine sample will be collected with the ThinPrep (Hologic) device. The total volume of the cell suspension obtained will be 20 mL. The samples will be transported to the accredited central laboratory for genotyping by special courier at −20° C. The service provider will take 4mL of the sample for HPV genotyping, the remaining volume of the sample (16mL) will be frozen at −20° C. According to the results of the HPV genotyping, the laboratory destroys the samples that are negative for HR-HPV (sending a certificate of destruction to Sponsor) stored the HR-HPV positive samples at −20° C., the samples will be transported in group to Genomic Vision at −20° C. upon agreement. Genomic Vision performs the analysis by Molecular Combing on the HR-HPV positive samples. Genomic Vision stores the rest of the samples for which Molecular Combing analysis has been performed for a period of 10 years after the end of the study (biological collection). Sponsor is responsible for the destruction of these samples by requesting a certificate of destruction.

Cervical uterine sample management at M6, M18, M30. Cervical-uterine sample collection will be performed with the ThinPrep (Hologic) device and will be shipped as usual to the site's cytology laboratory, which will perform the cytological analysis of the sample.

Molecular combing to search for HPV integration. The search for HPV genome integration involves 5 steps:

Step No. 1: DNA Extraction. The cells from the biological sample to be analysed are placed in a block of agarose called a “plug,” in which several enzyme treatment stages lead to sample lysis and the elimination of all proteins attached to the DNA (histones, transcription factors, etc.). The agarose is then digested to obtain naked DNA in solution.

Step No. 2: Combing. A glass slide coated with a fine layer of silane is dipped into the DNA solution. The double-stranded DNA is fixed irreversibly on the silanized slide at one and/or the other of the two ends by hydrophobic interactions. The glass slide is then removed from the solution vertically at a constant speed of 300 μm/s. The force stretches the DNA is applied exclusively at the meniscus and leads to a uniform stretching of the DNA molecules regardless of their length. The DNA strands are thus aligned parallel to each other and form a mat on the slide, which includes, according to its density, around 100 complete genomes.

Step No. 3: Hybridisation. The stretched DNA molecules are irreversibly fixed on the glass slide after combing; however, they remain accessible to additional DNA sequences of probes covering HPV genomes 16, 18, 31, 33, 45, 35, 39, 51, 52, 56, 58, 59, 66 and 68. These probes are obtained by random priming allowing the incorporation of modified nucleotides in the sequences that are detected with the help of specific antibodies of each modified nucleotide coupled with fluorochromes, thus generating a fluorescent signal.

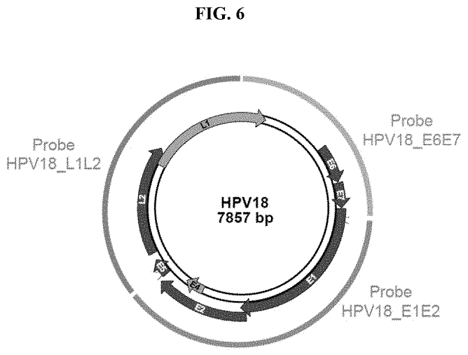

The HPV genomes studied are covered by 3 probes (FIG. 3): one specific to the viral genome region containing genes L1 and L2, the second specific to the region containing viral genes El and E2, and the third to the region containing viral oncogenes E6 and E7. The probes corresponding to regions L1L2 and E1E2 will be displayed in blue and the one corresponding to region E6E7 in cyan (green+blue). In addition, probes are generated that cover a reference locus of the host DNA, in order to quantify the number of host genomes combed and standardize HPV integrations by patient cell. These reference signals are displayed in red. See FIG. 6.

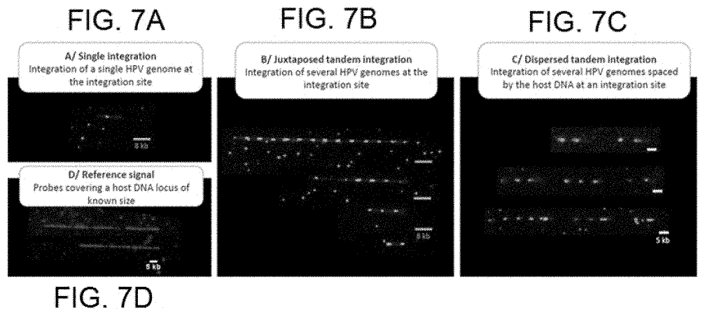

Step No. 4: Acquisition offluorescent signals. Image acquisition is performed by epifluorescence microscopy. The slide is lit by wavelengths corresponding to the various fluorochromes by means of filtered light (excitation spectrum) and the re-emitted fluorescent light is captured in the appropriate wavelengths (emission spectrum). In order to acquire the images, Genomic Vision has a scanner that is able to divide the slide into several thousand visual fields and capture the images for each visual field. The images thus generated are analysed by software that allows the detection of the regions of interest containing a fluorescent signal of interest and their measurement; see FIG. 7.

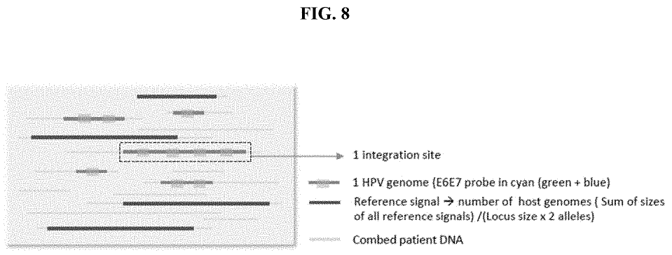

Step No. 5: Analysis. The HPV 16, 18, 31, 33, 45 35, 39, 51, 52, 56, 58, 59, 66 and 68 genomes are covered by probes: the probes covering L1L2 and E1E2 are displayed in blue and the one covering E6E7 in cyan (blue +green). The reference locus is displayed in red, see FIG. 8 which diagrams types of signals detected on the slides.

For each patient, the parameters which are noted during the Molecular Combing analysis are as follows:

Estimated number of host genomes combed on the slide: It is calculated by adding the sizes (in kb) of all the reference signals (red) visible on the analysed slide and dividing that sum by the theoretical size (in kb) of the locus, then by 2 (because there are 2 alleles per genome). In the case of smears that suggest a cancerous lesion, which have considerable genetic instability, we will start from the premise that this region has not been modified. Indeed, since the locus is especially poor in repeated sequences, there is a lower risk of being modified. This number of combed host genomes varies from one slide to another, according to the amount of DNA extracted and the density of the combing. It therefore serves to standardize the results.

Number of HPV integration sites on the slide. It corresponds to the number of HPV signals (blue) detected on the slide. In the example in the figure above, we can count 5.

Size in kilobases (kb) of the HPV genome integrations: This corresponds to the size of the HPV signals (blue) measured in kb.

Number of integrated HPV genomes at each integration site. This corresponds to the number of cyan signals visible for each blue HPV signal. For example, in the figure above, the framed HPV signal has 4 integrated HPV genomes at that site.

The integration profile will be defined by the following variables, whose values are deduced directly from the data stated above: The number of HPV integration sites per patient genome, the average number of integrated HPV genomes per integration site (or the average size of the integrations), the maximum number of integrated HPV genomes per integration site (or the maximum size of the integrations), the minimum number of integrated HPV genomes per integration site (or the minimum size of the integration sites), the number of integrated HPV genomes per patient genome.

These values are calculated for all the HPV signals (blue) detected and then only for the HPV signals (blue) greater than 10 kb. This selection of signals allow the elimination of potential contamination by episomal viral forms that are linearized during handling, allowing these forms to be combed by their free ends.

Centralized reading of biopsies and cones. With regard to the histology samples taken (biopsy in the case of a visible lesion and cone samples if the lesion is treated), the histological analysis with a centralised reading of the histology samples is performed by an accredited laboratory. For this purpose, the samples (blocks of tissues or slides) is sent by the research site to this laboratory for assessment, which is also perform immunohistochemical staining tests for p16 and Ki67 transformation markers. The samples are archived by the laboratory for 10 years (blocks of tissues) and 5 years (slides).

Centralized reading of cervical photographs. The photographs of the cervix are taken at 3 successive times during the colposcopic examination: Exam without preparation with an optional photograph taken with a green filter to analyse the epithelium and the vessels; Exam after applying 3% acetic acid after waiting from 30 seconds to 1 minute to allow the identification of the squamocolumnar junction and the search for acidophilus; Exam after applying Lugol's solution. A colposcopic diagram (optional) based on the findings after applying acetic acid allows visualization of the squamocolumnar junction, the extent of the acidophilus and, especially, the exact location and number of the biopsy or biopsies. A centralised reading of the photographs of the cervix are performed by 2 experienced gynaecologists who are independent of the study. The colposcopic situation is determined for each photograph.

A sample research plan is described below.

| | Baseline | Follow-up | Follow-up | Follow-up | Follow-up | Follow-up | Follow-up |
|---|---|---|---|---|---|---|---|
| | Visit | Visit | Visit | Visit | Visit | Visit | Visit |
| | M 0 | M 6 | M 12 | M 18 | M 24 | M 30 | M 36 |
| Consent | X | | | | | | |
| Urine pregnancy test | X | X | X | X | X | X | X |
| Collection of medical | X | | | | | | |
| history and lifestyle info | | | | | | | |
| Cervical uterine smear | X | X | X | X | X | X | X |
| Cytological analysis | | X | | X | | X | |
| HPV genotyping | X | | X | | X | | X |
| Molecular combing¹ | X | | X | | X | | X |
| Colposcopy | X | | X | | X | | X |
| Photograph of the cervix | X | | X | | X | | X |
| Cervical biopsies + centralised | X | | X | | X | | X |
| histology analysis² | | | | | | | |

¹in high-risk HPV positive patients
²as recommended

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Throughout this specification and the claims which follow, unless the context requires otherwise, the word “comprise”, and variations such as “comprises” and “comprising” means various components can be co jointly employed in the methods and articles (e.g., compositions and apparatuses including device and methods). For example, the term “comprising” will be understood to imply the inclusion of any stated elements or steps but not the exclusion of any other elements or steps.

As used herein in the specification and claims, including as used in the examples and unless otherwise expressly specified, all numbers may be read as if prefaced by the word “substantially”, “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), +/−15% of the stated value (or range of values), +/−20% of the stated value (or range of values), etc. Any numerical range recited herein is intended to include all sub-ranges subsumed therein.

### REFERENCES

Choo, K. B., C. C. Pan and S. H. Han (1987). “Integration of human papillomavirus type 16 into cellular DNA of cervical carcinoma: preferential deletion of the E2 gene and invariable retention of the long control region and the E6/E7 open reading frames.” Virology 161 (1): 259-261.

Cricca, M., A. M. Morselli-Labate, S. Venturoli, S. Ambretti, G. A. Gentilomi, G. Gallinella, S. Costa, M. Musiani and M. Zerbini (2007). “Viral DNA load, physical status and E2/E6 ratio as markers to grade HPV16 positive women for high-grade cervical lesions.” Gynecol Oncol 106 (3): 549-557.

Crosbie, E. J., M. H. Einstein, S. Franceschi and H. C. Kitchener (2013). “Human papillomavirus and cervical cancer.” Lancet 382 (9895): 889-899.

Dyson, N., P. M. Howley, K. Munger and E. Harlow (1989). “The human papilloma virus-16 E7 oncoprotein is able to bind to the retinoblastoma gene product.” Science 243 (4893): 934-937.

Ferber, M. J., E. C. Thorland, A. A. Brink, A. K. Rapp, L. A. Phillips, R. McGovern, B. S. Gostout, T. H. Cheung, T. K. Chung, W. Y. Fu and D. I. Smith (2003). “Preferential integration of human papillomavirus type 18 near the c-myc locus in cervical carcinoma.” Oncogene 22 (46): 7233-7242.

Gradissimo Oliveira, A., C. Delgado, N. Verdasca and A. Pista (2013). “Prognostic value of human papillomavirus types 16 and 18 DNA physical status in cervical intraepithelial neoplasia.” Clin Microbiol Infect 19 (10): E447-450.

Herrick, J., C. Conti, S. Teissier, F. Thierry, J. Couturier, X. Sastre-Garau, M. Favre, G. Orth and A. Bensimon (2005). “Genomic organization of amplified MYC genes suggests distinct mechanisms of amplification in tumorigenesis.” Cancer Res 65 (4): 1174-1179.

Hopman, A. H., F. Smedts, W. Dignef, M. Ummelen, G. Sonke, M. Mravunac, G. P. Vooijs, E. J. Speel and F. C. Ramaekers (2004). “Transition of high-grade cervical intraepithelial neoplasia to micro-invasive carcinoma is characterized by integration of HPV 16/18 and numerical chromosome abnormalities.” J Pathol 202 (1): 23-33.

Hu, Z., D. Zhu, W. Wang, W. Li, W. Jia, X. Zeng, W. Ding, L. Yu, X. Wang, L. Wang, H. Shen, C. Zhang, H. Liu, X. Liu, Y. Zhao, X. Fang, S. Li, W. Chen, T. Tang, A. Fu, Z. Wang, G. Chen, Q. Gao, S. Li, L. Xi, C. Wang, S. Liao, X. Ma, P. Wu, K. Li, S. Wang, J. Zhou, J. Wang, X. Xu, H. Wang and D. Ma (2015). “Genome-wide profiling of HPV integration in cervical cancer identifies clustered genomic hot spots and a potential microhomology-mediated integration mechanism.” Nat Genet 47 (2): 158-163.

Huang, L. W., S. L. Chao and B. H. Lee (2008). “Integration of human papillomavirus type-16 and type-18 is a very early event in cervical carcinogenesis.” J Clin Pathol 61 (5): 627-631.

Jeon, S., B. L. Allen-Hoffmann and P. F. Lambert (1995). “Integration of human papillomavirus type 16 into the human genome correlates with a selective growth advantage of cells.” J Virol 69 (5): 2989-2997.

Jeon, S. and P. F. Lambert (1995). “Integration of human papillomavirus type 16 DNA into the human genome leads to increased stability of E6 and E7 mRNAs: implications for cervical carcinogenesis.” Proc Natl Acad Sci USA 92 (5): 1654-1658. Klaes, R., S. M. Woerner, R. Ridder, N. Wentzensen, M. Duerst, A. Schneider, B. Lotz, P. Melsheimer and M. von Knebel Doeberitz (1999). “Detection of high-risk cervical intraepithelial neoplasia and cervical cancer by amplification of transcripts derived from integrated papillomavirus oncogenes.” Cancer Res 59 (24): 6132-6136.

Kulmala, S. M., S. M. Syrjanen, U. B. Gyllensten, I. P. Shabalova, N. Petrovichev, P. Tosi, K. J. Syrjanen and B. C. Johansson (2006). “Early integration of high copy HPV16 detectable in women with normal and low grade cervical cytology and histology.” J Clin Pathol 59 (5): 513-517.

Lebofsky, R. and A. Bensimon (2003). “Single DNA molecule analysis: applications of molecular combing.” Brief Funct Genomic Proteomic 1 (4): 385-396.

Luft, F., R. Klaes, M. Nees, M. Durst, V. Hellmann, P. Melsheimer and M. von Knebel Doeberitz (2001). “Detection of integrated papillomavirus sequences by ligationmediated PCR (DIPS-PCR) and molecular characterization in cervical cancer cells.” Int J Cancer 92 (1): 9-17.

Marongiu, L., A. Godi, J. V. Parry and S. Beddows (2014). “Human Papillomavirus 16, 18, 31 and 45 viral load, integration and methylation status stratified by cervical disease stage.” BMC Cancer 14: 384.

Peitsaro, P., B. Johansson and S. Syrjanen (2002). “Integrated human papillomavirus type 16 is frequently found in cervical cancer precursors as demonstrated by a novel quantitative real-time PCR technique.” J Clin Microbiol 40 (3): 886-891.

Peter, M., C. Rosty, J. Couturier, F. Radvanyi, H. Teshima and X. Sastre-Garau (2006). “MYC activation associated with the integration of HPV DNA at the MYC locus in genital tumors.” Oncogene 25 (44): 5985-5993.

Pett, M. and N. Coleman (2007). “Integration of high-risk human papillomavirus: a key event in cervical carcinogenesis?” J Pathol 212 (4): 356-367.

Raybould, R., A. Fiander, G. W. Wilkinson and S. Hibbitts (2014). “HPV integration detection in CaSki and SiHa using detection of integrated papillomavirus sequences and restriction-site PCR.” J Virol Methods 206: 51-54.

Sabol I, Salakova M, Smahelova J, Pawlita M, Schmitt M, Gasperov NM, Grce M, Tachezy R. (2008). “Evaluation of different techniques for identification of human papillomavirus types of low prevalence.” J Clin Microbiol. 46 (5):1606-13.

Scheffner, M., B. A. Werness, J. M. Huibregtse, A. J. Levine and P. M. Howley (1990). “The E6 oncoprotein encoded by human papillomavirus types 16 and 18 promotes the degradation of p53.” Cell 63 (6): 1129-1136.

Van Tine, B. A., J. Knops, T. R. Broker, L. T. Chow and P. T. Moen, Jr. (2001). “In situ analysis of the transcriptional activity of integrated viral DNA using tyramideFlSH.” Dev Biol (Basel) 106: 381-385.

Vega-Pena, A., B. Illades-Aguiar, E. Flores-Alfaro, E. Lopez-Bayghen, M. A. LeyvaVazquez, E. Castaneda-Saucedo and C. Alarcon-Romero Ldel (2013). “Risk of progression of early cervical lesions is associated with integration and persistence of HPV-16 and expression of E6, Ki-67, and telomerase.” J Cytol 30 (4): 226-232.

Vinokurova, S., N. Wentzensen, I. Kraus, R. Klaes, C. Driesch, P. Melsheimer, F. Kisseljov, M. Durst, A. Schneider and M. von Knebel Doeberitz (2008). “Typedependent integration frequency of human papillomavirus genomes in cervical lesions.” Cancer Res 68 (1): 307-313.

von Knebel Doeberitz, M., T. Bauknecht, D. Bartsch and H. zur Hausen (1991). “Influence of chromosomal integration on glucocorticoid-regulated transcription of growth-stimulating papillomavirus genes E6 and E7 in cervical carcinoma cells.” Proc Nati Acad Sci U S A 88 (4): 1411-1415.

Wentzensen, N., R. Ridder, R. Klaes, S. Vinokurova, U. Schaefer and M. Doeberitz (2002). “Characterization of viral-cellular fusion transcripts in a large series of HPV16 and 18 positive anogenital lesions.” Oncogene 21 (3): 419-426.

Wentzensen, N., S. Vinokurova and M. von Knebel Doeberitz (2004). “Systematic review of genomic integration sites of human papillomavirus genomes in epithelial dysplasia and invasive cancer of the female lower genital tract.” Cancer Res 64 (11): 3878-3884.

Ziegert, C., N. Wentzensen, S. Vinokurova, F. Kisseljov, J. Einenkel, M. Hoeckel and M. von Knebel Doeberitz (2003). “A comprehensive analysis of HPV integration loci in anogenital lesions combining transcript and genome-based amplification techniques.” Oncogene 22 (25): 3977-3984.

Zubillaga-Guerrero, M. I., B. Illades-Aguiar, M. A. Leyva-Vazquez, E. Flores-Alfaro, E. Castaneda-Saucedo, J. F. Munoz-Valle and L. C. Alarcon-Romero (2013). “The integration of HR-HPV increases the expression of cyclins A and E in cytologies with and without low-grade lesions.” J Cytol 30(1): 1-7. zur Hausen, H. (2002). “Papillomaviruses and cancer: from basic studies to clinical application.” Nat Rev Cancer 2 (5): 342-350.

All publications and patent applications mentioned in this specification are herein incorporated by reference in their entirety to the same extent as if each individual publication or patent application was specifically and individually indicated to be incorporated by reference, especially referenced is disclosure appearing in the same sentence, paragraph, page or section of the specification in which the incorporation by reference appears.

The citation of references herein does not constitute an admission that those references are prior art or have any relevance to the patentability of the technology disclosed herein. Any discussion of the content of references cited is intended merely to provide a general summary of assertions made by the authors of the references, and does not constitute an admission as to the accuracy of the content of such references.

1-20. (canceled)

21. A method for assessing a risk of having or developing cervical cancer in patient comprising:
isolating genomic DNA from cervical cells of the patient,
molecularly combing the isolated genomic DNA,
contacting the combed genomic DNA with fluorescent probes that bind to HPV DNA in the genomic DNA under conditions suitable for hybridization between HPV DNA in the genomic DNA and the fluorescent probes, thereby providing fluorescent probe-hybridized genomic DNA:
detecting from probe-hybridized genomic DNA a number of HPV integration sites per patient genome, an average number of integrated HPV genomes per integration site, an average size of HPV integrations into the genomic DNA, a minimum number of integrated HPV genomes per HPV integration site, and/or a minimum size of the HPV integration sites, thereby providing an integration profile for the patient,
comparing the integration profile of the patient to that of a control patient having a normal cervix; and
performing a colposcopy, cervical biopsy, and/or centralized histology analysis when the number of HPV integration sites per patient genome, the average number of integrated HPV genomes per integration site, the average size of the HPV integrations into the genomic DNA, the minimum number of integrated HPV genomes per HPV integration site, and/or the minimum size of the HPV integration sites is greater than that in the control patient.

22. The method of claim 21, further comprising treating the patient for a cervical disorder, after performing the colposcopy, cervical biopsy, and/or centralized histology analysis.

23. The method of claim 21, wherein the patient has not be diagnosed with cervical cancer.

24. The method of claim 21, wherein the patient has a precancerous lesion.

25. The method of claim 21, wherein the patient or subject has a cervical dysplasia or has a positive PAP test.

26. The method of claim 21, wherein the patient has been infected with human immunodeficiency virus (HIV), is immunosuppressed, has been exposed to diethylstilbestrol before birth, or is or has been treated for a precancerous cervical lesion or cervical cancer.

27. The method of claim 21, wherein the patient has anal, vaginal, vulvar, penile or oropharyngeal cancer.

28. The method of claim 21, wherein the control patient has a normal cervix.

29. The method of claim 21, wherein the control patient has no lesions and substantially no antibody titer to HPV.

30. The method of claim 21, wherein said control subject or control patient is the same subject or patient at an earlier point in time.

31. The method of claim 21, wherein the fluorescent probes hybridize to HPV 16, 18, 31, 33, 45 35, 39, 51, 52, 56, 58, 59, 66 or 68 DNA.

32. The method of claim 21, wherein the fluorescent probes hybridize to HPV 6 or 11.

33. The method of claim 21, wherein the integration profile is determined only for HPV hybridizations greater than 10 kb.

34. The method of claim 21, wherein the integration profile is determined from the number of HPV integration sites per patient genome.

35. The method of claim 21, wherein the integration profile is determined from the average number of integrated HPV genomes per integration site or from the minimum number of integrated HPV genomes per HPV integration site.

36. The method of claim 21, wherein the integration profile is determined from the average size of the HPV integrations into the genomic DNA or from the minimum size of the HPV integration sites.

37. The method of claim 21, wherein the integration profile is determined from the minimum number of integrated HPV genomes per HPV integration site, or the minimum size of the HPV integration sites.

38. The method of claim 22, wherein said treating comprises treating a low grade precancerous lesion.

39. The method of claim 22, wherein said treating comprises treating a high-grade precancerous lesion.

40. The method of claim 22, wherein said treating comprises a pharmaceutical treatment.
